(19) United States
(12) Patent Application Publication
(10) Pub. No.: US 2026/0331250 A1
(43) Pub. Date: Oct. 22, 2026

(54) CLINICAL THERAPEUTIC DRUG PREDICTION AND RECOMMENDATION SYSTEM AND METHOD FOR EVALUATING EFFICACY OF SECOND-GENERATION HORMONE DRUG IN TREATMENT OF PROSTATE CANCER

(71) Applicant: Kaohsiung Medical University, Kaohsiung City (TW)

(72) Inventors: Shu-Pin Huang, Kaohsiung City (TW); Chia-Yang Li, Kaohsiung City (TW); Deng-Neng Chen, Pingtung County (TW); Wei-Ju Chung, Kaohsiung City (TW)

(73) Assignee: Kaohsiung Medical University, Kaohsiung City, TW (TW)

(21) Appl. No.: 19/728,350

(22) Filed: Jul. 2, 2026

Related U.S. Application Data
(63) Continuation-in-part of application No. 18/578,721, filed on Jul. 20, 2022.
(60) Provisional application No. 63/224,875, filed on Jul. 23, 2021.

(51) Int. Cl. G16H 20/10; G16H 10/40; G16H 40/63

(52) U.S. Cl. CPC G16H 20/10; G16H 10/40; G16H 40/63

(57) ABSTRACT

A clinical therapeutic drug prediction and recommendation system—for evaluating the efficacy of a second-generation hormone drug in treatment of prostate cancer is provided, including an input device for receiving an ex vivo biological sample and generating a physiological signal; a computer device including a processor for receiving the physiological signal, the processor including an analysis module which compares the physiological signal with—gene sets of patient suitable for a first drug and a second drug treatment respectively, and then respectively obtains a first score and a second score corresponding to the first drug and the second drug by a calculation method, the scores being further compared by a comparison module, when the comparison result shows that the first score is greater than the second score, an indication of recommending the first drug to the individual being given; an indication of recommending the second drug to the individual being given.

### CROSS REFERENCE TO RELATED APPLICATIONS

This application is a Continuation-in-Part application of the pending U.S. patent application Ser. No. 18/578,721 filed on Jan. 12, 2024, which is a national stage application, filed under 35 U.S.C. § 371, of International Patent Application No. PCT/CN2022/106720 filed on Jul. 20, 2022, which claims the benefit of U.S. Provisional Patent Application No. 63/224,875, filed Jul. 23, 2021, each of which is incorporated by reference herein in its entirety. Although incorporated by reference in its entirety, no arguments or disclaimers made in the parent application apply to this divisional application. Any disclaimer that may have occurred during the prosecution of the above-referenced application(s) is hereby expressly rescinded. Consequently, the Patent Office is asked to review the new set of claims in view of the entire prior art of record and any search that the Office deems appropriate.

### FIELD OF THE INVENTION

The present invention relates to a clinical therapeutic drug prediction and recommendation system, in particular a clinical therapeutic drug prediction and recommendation system for evaluating the efficacy of second-generation hormone drugs in the treatment of prostate cancer.

### BACKGROUND OF THE INVENTION

According to the statistics of 2019, prostate cancer ranked sixth in Taiwan's cancer death ranking. It is well known that many prostate cancer patients are resistant to first-generation hormone therapeutic drugs, and because chronic inflammation causes induction, development and recurrence of prostate cancer, patients must subsequently receive second-generation hormone therapy.

The second-generation hormone therapeutic drugs are the first choice for patients' subsequent androgen deprivation therapy (ADT). There are currently two types of second-generation hormone therapeutic drugs. One is a compound of androgen synthesis inhibitor Abiraterone, the other is a compound of androgen receptor blocker Enzalutamide, Apalutamide and Darolutamide. These two types of second-generation hormone therapies have therapeutic effects on treating metastatic castration-resistant prostate cancer. The two types of drugs both are health insurance drugs, but they cost a lot. Abiraterone costs about NT$90,000 per month, and Enzalutamide costs about NT$100,000 per month.

It may prolong the survival time of the patients when the patients randomly take one of the second-generation hormone drugs, but the efficacy of various second-generation hormone therapeutic drugs for different patients is largely different. If the efficacy is good, it can stably control cancer progression for several years. If the efficacy is poor, the drug resistance will develop after a few months of treatment. After the occurrence of drug resistance, the patients only are treated with follow-up chemotherapy, and their lives may come to an end in a short time. However, there is currently no technology, criterion or clinical literature to help doctors determine which patients are suitable for which drugs. Therefore, how to choose the appropriate second-generation hormone therapeutic drugs for the patients has become a major challenge in prostate cancer treatment.

### SUMMARY OF THE INVENTION

The present invention relates to a system for indicating the use of second-generation hormone drugs in the treatment of prostate cancer, comprising: an input device for receiving ex vivo biological samples obtained from a subject and generating at least one physiological signal (N) of the ex vivo biological samples; and a computer device connected to the input device, wherein the computer device comprising: a processor for receiving the at least one physiological signal, which further comprises: an analysis module for comparing the at least one physiological signal with a first-level gene set (X1) of patients suitable for a first drug treatment and a second-level gene set (X2) of the patients suitable for the first drug treatment, respectively, then respectively obtain a comparison value of (N, X1) and a comparison value of (N, X2), a first score (S1) corresponding to the first drug treatment in the patients is calculated by using a calculation method based on the comparison value of (N, X1) and the comparison value of (N, X2), and comparing the at least one physiological signal with a first-level gene set (Y1) of patients suitable for a second drug treatment and a second-level gene set (Y2) of the patients suitable for the second drug treatment, respectively, then respectively obtain a comparison value of (N, Y1) and a comparison value of (N, Y2), a second score (S2) corresponding to the second drug treatment in the patients is calculated by using the calculation method based on the comparison value of (N, Y1) and the comparison value of (N, Y2); a comparison module connected to the analysis module for comparing the first score (S1) and the second score (S2) to obtain a comparison result; and a recommendation module connected to the comparison module for receiving the comparison result and outputting an indication, wherein an indication for recommending the subject to take the first drug is given when the first score (S1) is higher than the second score (S2), or an indication for recommending the subject to take the second drug is given when the first score (S1) is lower than the second score (S2).

### DETAILED DESCRIPTION OF THE INVENTION

The present invention provides a system for indicating the use of an appropriate second-generation hormone drugs in the treatment of prostate cancer, comprising: an input device for receiving ex vivo biological samples obtained from a subject suffering from prostate cancer, and analyzing the ex vivo biological samples to generate at least one physiological signal (N) comprising genetic information of the treated subject suffering from prostate cancer; and a computer device connected to the input device, wherein the computer device comprises: a processor for receiving the at least one physiological signal (N), wherein the processor further comprises an analysis module for executing a calculation method to generate a first score (S1) and a second score (S2) by: comparing the at least one physiological signal (N) with a first-level gene set (X1) of patients suitable for a first second-generation drug treatment to obtain a comparison value of (N, X1) and comparing the at least one physiological signal (N) with a second-level gene set (X2) of the patients suitable for the first second-generation drug treatment to obtain a comparison value of (N, X2), then obtaining a first score (S1) by using a calculation method, and comparing the at least one physiological signal (N) with a first-level gene set (Y1) of patients suitable for a second second-generation drug treatment to obtain a comparison value of (N, Y1) and comparing the at least one physiological signal (N) with a second-level gene set (Y2) of the patients suitable for the second second-generation drug treatment to obtain a comparison value of (N, Y2), then obtaining a second score (S2) by using the calculation method; a comparison module for comparing the first score (S1) and the second score (S2) to obtain a comparison result; and a recommendation module for receiving the comparison result and outputting an indication, wherein the recommendation module gives an indication to recommend the subject suffering from prostate cancer to take the first second-generation hormone drug when the first score (S1) is higher than the second score (S2), or gives an indication to recommend the subject suffering from prostate cancer to take the second second-generation hormone drug when the first score (S1) is lower than the second score (S2).

In the present invention, the recommendation module further connected to an electronic medical record system to output a prescription to the electronic medical record system according to the given indication.

In the present invention, the recommendation module further connected to an automated dispensing cabinet connected to dispense the second-generation hormone drug according to the given indication.

In the present invention, the computer device further comprises a display device for displaying the recommended indications.

In the present invention, the computer device further comprises a storage module for storing the at least one physiological signal, the comparison results and the recommended indications.

In a preferred embodiment of the present invention, the at least one physiological signal comprises genetic information of the ex vivo biological samples.

In a preferred embodiment of the present invention, the first second-generation hormone drug is a compound of an androgen receptor blocker, and the second second-generation hormone drug is a compound of an androgen synthesis inhibitor.

In the present invention, the calculation method comprises at least one algorithm.

In the present invention, the at least one algorithm comprises Euclidian Distance, Manhattan Distance, Hamming Distance, Cosine similarity algorithm or Jaccard Similarity (JS) coefficient.

In a preferred embodiment of the present invention, the algorithm is Jaccard Similarity coefficient.

In a preferred embodiment of the present invention, the Jaccard Similarity coefficient is used to calculate the comparison value of (N, X1), the comparison value of (N, X2), the comparison value of (N, Y1) and the comparison value of (N, Y2).

In the present invention, preferably, the calculation method further comprises a first weight value (W1) corresponding to the comparison value of (N, X1) or the comparison value of (N, Y1), and a second weight value (W2) corresponding to the comparison value of (N, X2) or the comparison value of (N, Y2).

In the present invention, preferably, the weight value ranges from 0 to 1.

The present invention further provides a method for indicating the use of an appropriate second-generation hormone drugs in the treatment of prostate cancer, comprising: receiving ex vivo biological samples obtained from a subject suffering from prostate cancer by an input device and analyzing the ex vivo biological samples to generate at least one physiological signal (N) comprising genetic information of the subject suffering from prostate cancer and sending the at least one physiological signal (N) to a processor; comparing the at least one physiological signal (N) with a first-level gene set (X1) of patients suitable for a first second-generation hormone drug treatment to obtain a comparison value of (N, X1) and comparing the at least one physiological signal (N) with a second-level gene set (X2) of the patients suitable for the first second-generation hormone drug treatment to obtain a comparison value of (N, X2) by an analysis module of the processor, then applying a first weight value (W1) to the comparison value of (N, X1) and a second weight value (W2) to the comparison value of (N, X2) to obtain the first score (S1) by using a calculation method, and comparing the at least one physiological signal (N) with a first-level gene set (Y1) of patients suitable for a second second-generation hormone drug treatment to obtain a comparison value of (N, Y1) and comparing the at least one of the physiological signals (N) with a second-level gene set (Y2) of the patients suitable for the second second-generation hormone drug treatment to obtain a comparison value of (N, Y2) by the analysis module of the processor, then applying the first weight value (W1) to the comparison value of (N, Y1) and the second weight value (W2) to the comparison value of (N, Y2) to obtain the second score (S2) by using the calculation method; comparing the first score (S1) and the second score (S2) to generate a comparison result by using a comparison module of the processor; and receiving the comparison result and giving an indication by an recommendation module to recommend the subject suffering from prostate cancer to take the first second-generation hormone drug when the first score (S1) is higher than the second score (S2), or gives an indication to recommend the subject suffering from prostate cancer to take the second second-generation hormone drug when the first score (S1) is lower than the second score (S2).

In the present invention, preferably, the method further comprises a step to administer the recommended second-generation hormone drug to the untreated subject suffering from prostate cancer for prostate cancer treatment.

### BRIEF DESCRIPTION OF THE DRAWINGS

FIGS. 1A to 1B show schematic diagrams of a clinical therapeutic drug prediction and recommendation system of the present invention. FIG. 1A is a schematic diagram without a storage module. FIG. 1B is a schematic diagram containing a storage module.

FIG. 2 shows a flowchart of a clinical therapeutic drug prediction and recommendation method for evaluating the efficacy of the second-generation hormone drug in treatment of prostate cancer in the present invention.

FIGS. 3A to 3C show descriptions of the calculation method of the present invention. FIG. 3A is a schematic diagram of an analysis algorithm based on the compound of androgen synthesis inhibitor Abiraterone. FIG. 3B is a schematic diagram of a analysis algorithm based on the compound of androgen receptor blocker Enzalutamide. FIG. 3C is a procedure for comparing the results of the patient responded to various drugs.

### EMBODIMENTS

The following embodiments are not intended to limit the scope of the present invention, but to make the above and other objects, features and advantages of the present invention more obvious.

Second-generation hormone drugs, including androgen receptor blocker or androgen synthesis inhibitor, are currently used for androgen deprivation therapy (ADT). Since second-generation hormone drugs are currently administered randomly to prostate cancer patients clinically, this leads to a risk of poor prognosis. Based on precision medicine, the invention aims to find a solution for precisely delivering suitable second-generation hormones to prostate cancer patients.

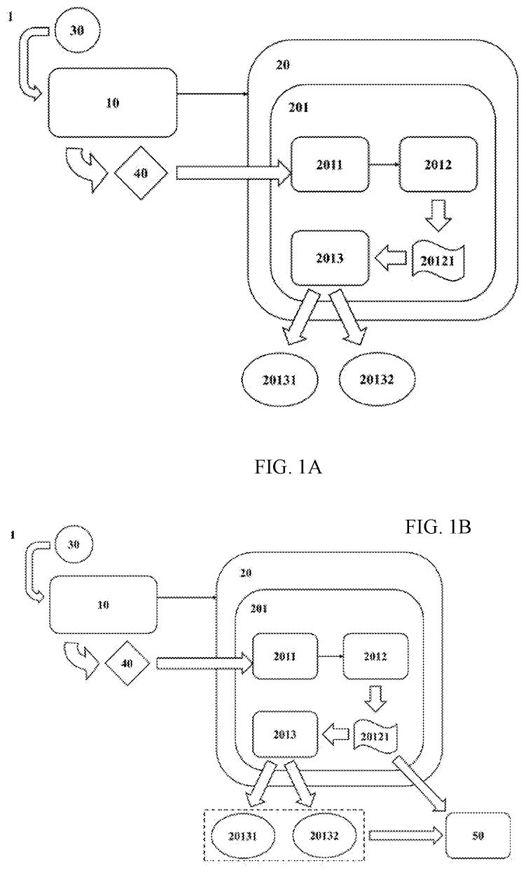

A system 1 for indicating the use of an appropriate second-generation hormone drugs in the treatment of the present invention is shown in FIG. 1A, which was used to evaluate the efficacy of second-generation hormone drugs in the patients suffering from prostate cancer and seeking second-generation hormone drugs treatment and to indicate an appropriate second-generation hormone drugs to those patients suffering from prostate cancer during the treatment of prostate cancer, comprising: an input device 10 for receiving ex vivo biological samples 30 obtained from the patients suffering from prostate cancer and seeking second-generation hormone drugs treatment, and analyzing the ex vivo biological samples to generate at least one physiological signal (N) 40 comprising genetic information of the patients suffering from prostate cancer and seeking second-generation hormone drugs treatment; and a computer device 20 connected to the input device 10, wherein the computer device 20 comprised: a processor 201 for receiving the at least one physiological signal (N) 40, wherein the processor 201 further comprises an analysis module 2011 for comparing the at least one physiological signal (N) 40 with a first-level gene set (X1) of patients suitable for a first second-generation hormone drug treatment to obtain comparison value of (N, X1) and comparing the at least one physiological signal (N) 40 with a second-level gene set (X2) of the patients suitable for the first second-generation hormone drug treatment to obtain comparison value of (N, X2), then obtaining the first score (S1) by using a calculation method, and comparing the at least one physiological signal (N) 40 with a first-level gene set (Y1) of patients suitable for a second second-generation hormone drug treatment to obtain a comparison value of (N, Y1) and comparing the at least one physiological signal (N) 40 with a second-level gene set (Y2) of the patients suitable for the second second-generation hormone drug treatment to obtain comparison value of (N, Y2), then obtaining a second score (S2) by using the calculation method; a comparison module 2012 connected to the analysis module for comparing the first score (S1) and the second score (S2) to obtain a comparison result 20121; and a recommendation module 2013 connected to the comparison module for receiving and analyzing the comparison result 20121, wherein the recommendation module gave an indication 20131 to recommend the patients suffering from prostate cancer and seeking second-generation hormone drugs treatment to take the first second-generation hormone drug when the first score (S1) is higher than the second score (S2), or gave an indication 20132 to recommend the patients suffering from prostate cancer and seeking second-generation hormone drugs treatment to take the second second-generation hormone drug when the first score (S1) is lower than the second score (S2).

For the purpose of precise drug treatments for prostate cancer, in a preferred manner, the recommendation module could connect to an electronic medical record (EMR) system to output a prescription to the electronic medical record system according to the given indication.

In another preferred manner, the recommendation module could further connect to an automated dispensing cabinet connected to dispense the second-generation hormone drug according to the given indication to the patients suffering from prostate cancer and seeking second-generation hormone drugs treatment, so that the patients could receive precise treatment of prostate cancer.

Further, as shown in FIG. 1B, the data or information could be transmitted to a storage module 50 of the computer device for storage, then the saved data or information could be used to perform algorithm calculations in the analysis module. After obtaining the data or information from the storage module, it was compared and calculated with the gene sets of patients suitable for different second-generation hormone drug treatments stored in the storage module or obtained by way of the network. Then, the results of the individual calculations were stored in the storage module, and displayed to the operator and the target patient by a display device (such as a screen, or monitor).

The present invention further comprised a display device. The display device could be a complete set of combination with the present invention or an independent external connection.

The input device of the present invention was a biochemical instrument well known to persons skilled in the art, including but not limited to: DNA sequencer, next-generation sequencer, biochemical serum analysis system, drug and special protein analyzer, electrolyte analyzer, glycated hemoglobin analyzer or osmotic pressure analyzer. In addition to receiving biological samples to be tested, the above biochemical instrument could be connected with the computer device by network, software and hardware integration for facilitating the performing analysis of the operator.

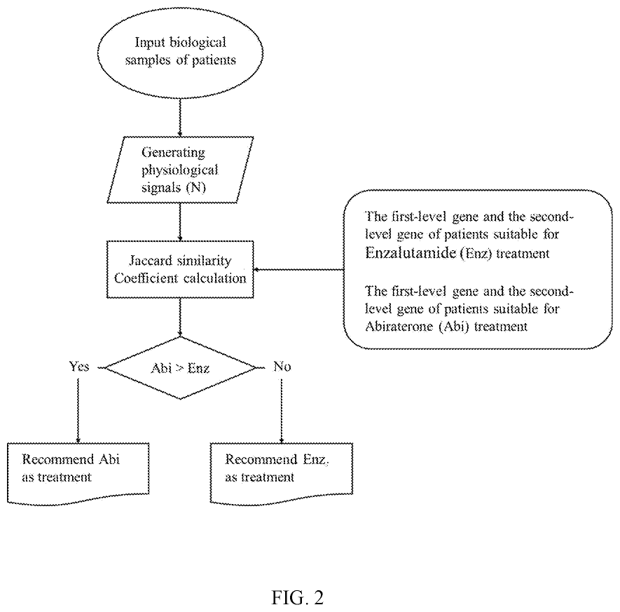

As shown in FIG. 2, a flowchart of a method of the present invention for indicating the use of an appropriate second-generation hormone drugs in the treatment of prostate cancer was depicted. At first, obtaining biological samples from a target patient suffering from prostate cancer and seeking second-generation hormone drugs treatment, wherein the biological samples could be blood, body fluids, hair, skin dander or other tissue fragments, and the biological sample was placed into the input device of the present invention to perform an input step, and the purpose was to be able to obtain the physiological signal (N) of the target patient by analyzing the biological samples, wherein the physiological signal (N) comprises genetic information of the target patient suffering from prostate cancer and seeking second-generation hormone drugs treatment in a preferred embodiment, in a preferred manner, the genetic information was the whole genes of the target patient. The genetic information was input into the processor of the computer device, and then the analysis step of physiological signal (N) was performed by the analysis module of the processor, and the at least one physiological signal (N) was compared with a first-level gene set (X1) of patients suitable for the first second-generation hormone drug treatment to obtain a comparison value of (N, X1), the at least one of the physiological signals (N) was compared with a set second-level gene set (X2) of the patients suitable for the first second-generation hormone drug treatment to obtain a comparison value of (N, X2), and then calculated the first score (S1) by Jaccard similarity coefficient and similarity weighting, and compared the at least one physiological signal (N) with a first-level gene set (Y1) of patients suitable for a second second-generation hormone drug treatment to obtain a comparison value of (N, Y1), and compared the at least one physiological signal (N) with a second-level gene set (Y2) of the patients suitable for the second second-generation hormone drug treatment to obtain a comparison value of (N, Y2), and then calculated the second score (S2) by Jaccard similarity coefficient and similarity weighting. Then, it compared the first score (S1) and the second score (S2) by the comparison module of the present invention, and the recommendation module of the present invention performed a recommendation step to give an indication to recommend the target patient suffering from prostate cancer and seeking second-generation hormone drugs treatment to take the first second-generation hormone drug when the first score (S1) is higher than the second score (S2), or the recommendation module gave an indication to recommend the target patient suffering from prostate cancer and seeking second-generation hormone drugs treatment to take the second second-generation hormone drug when the first score (S1) is lower than the second score (S2).

For a further precise drug treatment, an administration step was involved in the method for administration of an appropriate second-generation hormone therapeutic drugs to the target patient suffering from prostate cancer and seeking second-generation hormone drugs treatment could be performed according to the results above.

In one embodiment, the first second-generation hormone drug is a compound of an androgen receptor blocker, Enzalutamide (Enz) was used as example in the embodiment, and the second second-generation hormone drug is a compound of an androgen synthesis inhibitor, Abiraterone (Abi) was used as example in the embodiment.

In one embodiment, the genetic set collection of patients treated suitable for different second-generation hormone drugs could be obtained from the network or previous analysis records.

Further, the data or information could be transmitted to the storage module of the computer device for storage, the saved data or information then could be used to perform algorithmic calculations by the analysis module. After the data or information were obtained from the storage module, it was compared and calculated with the gene set of patients suitable for different second-generation hormone drug treatments stored in the storage module or obtained by way of network, and then the results of the individual calculation were stored in the storage module and displayed to the operator and the target patient by the display device (such as a screen, or monitor).

The calculation formula of Jaccard similarity coefficient used in the present invention was shown in the following Equation (I). Generally, it represented a definition for comparing the similarity and difference between finite sample sets: given two sets A, B, and the Jaccard similarity coefficient was defined as the ratio of the size of the intersection of A and B to the size of the union of A and B.

$$J ( A , B ) = \frac{\vert A ⋂ B \vert}{\vert A ⋃ B \vert} = \frac{\vert A ⋂ B \vert}{\vert A \vert + \vert B \vert - \vert A ⋂ B \vert} \qquad Equation ( I )$$

In the present invention, Jaccard similarity coefficient was used to calculate the comparison value of (N, X1), the comparison value of (N, X2), the comparison value of (N, Y1) and the comparison value of (N, Y2). Further, the weighting of the similarity was a first weight value (W1) corresponding to the comparison value of (N, X1) or the comparison value of (N, Y1), and a second weight value (W2) corresponding to the comparison value of (N, X2) or the comparison value of (N, Y2).

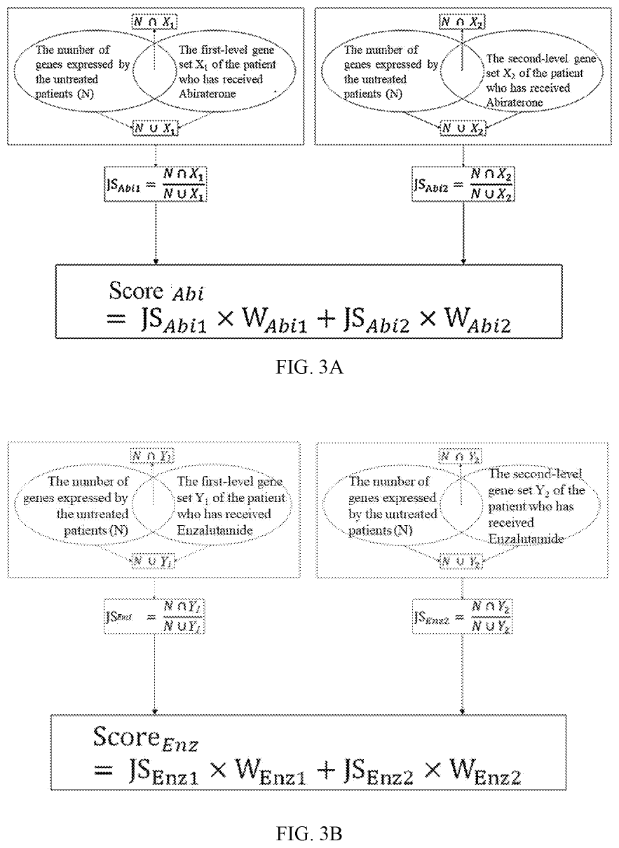

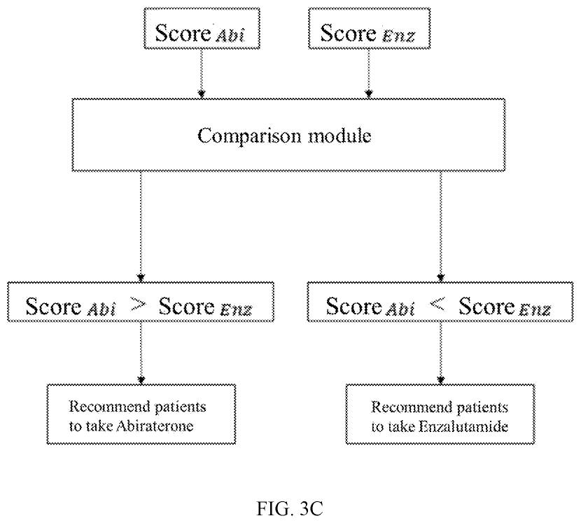

As shown in FIG. 3A, an analysis algorithm based on the compound of androgen synthesis inhibitor Abiraterone (Abi) was taken as an example. The present invention performed multiple calculations by the algorithm for comparison and calculation. Each time, the number of genes expressed by the untreated patients (N) was provided and calculated with the first-level gene set (X1) of the patient who had received Abiraterone by Jaccard Similarity to obtain JS_{Abi1}; then the number of genes expressed by untreated patients (N) was provided and calculated with the secondary gene set (X2) of patients who has received Abiraterone by Jaccard Similarity to obtain JS_{Abi2}; and finally according to the following Equation (II), different weight values W_{Abi1} and W_{Abi2} were given respectively, and added together to obtain Score_{Abi} which was used as the score of the patient responded to Abiraterone.

Score_{Abi}=JS_{Abi1}×W_{Abi1}+JS_{Abi2}×W_{Abi2} Equation (II)

As shown in Table 1, the first-level gene set (X1) regarding to Abiraterone for comparison comprises 11 genes, and the second-level gene set (X2) regarding to Abiraterone for comparison comprises 47 genes, wherein the genes of the first-level gene set (X1) were different from those of the second-level gene set (X2), and the weight values W_{Abi1} and W_{Abi2} were 1 and 0.8, respectively.

TABLE 1

| Gene | Number | |
|---|---|---|
| sets | of genes | Gene name |
| X1 | 11 | NR4A1, ARHGEF10L, TNNT1, CPAMD8, CYP2S1, |
| | | PLA2G7, CLEC10A, CD1C, ATF3, FCER1A, PLD4 |
| X2 | 47 | SHROOM1, OSM, CD83, STAB1, UCN, |
| | | AC106782.1, ANKRD28, CD69, FCHO2, CCR3, |
| | | CPA3, CCR1, IFNG, OXER1, GAS6, KCNC3, |
| | | ZNF385A, ELANE, BPI, FAM109A, LCN2, AZU1, |
| | | DEFA3, MZB1, PCSK6, KLF4, IFI27, SIGLEC1, |
| | | LGALS2, LRP1, PLXNB2, GATA2, FOSB, ZNF703, |
| | | HIST1H1C, NUCB2, JUN, C1orf54, CDC42EP1, |
| | | LILRB4, TUBB2A, CD24, RHOB, ITGB3, TGFBI, |
| | | AHSP, ZNF358 |

FIG. 3B showed a schematic diagram of analysis algorithm that use of the compound of androgen receptor blocker Enzalutamide (Enz) as an example. The present invention performed multiple calculations by the algorithm for comparison and calculation as the same as FIG. 3A. Taking this embodiment as an example, it performed two times calculations. Each time, the number of genes expressed by the untreated patients (N) were provided and calculated with the first-level gene set (Y1) of the patient who has received Enzalutamide by Jaccard Similarity to obtain JS_{Enz1}; then the number of genes expressed by untreated patients (N) were provided and calculated with the second-level gene set (Y2) of patients who have received Enzalutamide by Jaccard Similarity to obtain JS_{Enz2}; and finally according to the following equation (III), different weight values W_{Enz1} and W_{Enz2} were given respectively, and added together to obtain Score_{Enz} which was used as the score of the patient responded to Enzalutamide.

Score_{Enz}=JS_{Enz1}×W_{Enz1}+JS_{Enz2}×W_{Enz2} Equation (III)

Regarding the algorithm calculation flow of Enzalutamide, as shown in Table 2, the first-level gene set (Y1) comprises 5 genes, and the second-level gene set (Y2) comprises 165 genes, wherein the gene sequences of the first-level gene set (Y1) were different from those of the second gene set (Y2), and the weight values W_{Enz1} and W_{Enz2} were 1 and 0.8, respectively.

TABLE 2

| | Num- | |
|---|---|---|
| Gene | ber of | |
| sets | genes | Gene name |
| Y1 | 5 | TMF1, SLC4A1, ASH1L, ARMC2, STXBP5 |
| Y2 | 165 | SOD2, AQP9, FAM174A, SRSF11, ZBTB11, |
| | | CDC42EP3, CDK17, NFIL3, GCA, HBG2, HEATR5B, |
| | | MANF, TTC3, XAF1, APOBEC3A, STRN3, HBQ1, |
| | | WSB1, TNFAIP3, ZDHHC17, MYSM1, ZMAT1, |
| | | DDX3Y, CPEB4, ACSL1, FAM156A, ERBIN, WDR44, |
| | | HBB, SLC39A10, AL132780.3, FPR2, TREM1, RFX2, |
| | | ATRX, TNIK, PCMTD2, TMEM127, PLCG1, ZBTB38, |
| | | PHC3, ANKRD28, CXCR2, DYNC1LI2, RNF103, |
| | | EARS2, RNF19B, TLR8, HELZ2, ZNF518A, SLC38A2, |
| | | MPZL1, CRLF1, DNAJC25-GNG10, RBL2, G0S2, |
| | | CREBZF, LEMD3, TRAPPC10, MAP4K5, MGAM, |
| | | PIK3R4, SCYL2, FAM198B, SDCBP, EIF5, PIGA, |
| | | TAF1, KRR1, HIF1A, TLR4, GPD2, TCP11L2, PTP4A1, |
| | | SREK1, ARID4B, KCTD12, TNFAIP8L2, TOB1, HBA2, |
| | | BIRC3, PIKFYVE, UTRN, CDC14A, FAM76B, SENP7, |
| | | GPR171, NKTR, GOLGA8N, NFKBIZ, GNS, PTGS2, |
| | | ATXN7, ITGA6, PRF1, TNFRSF10C, RNPC3, KLF9, |
| | | FAM91A1, PDK4, KIAA1109, ZNF548, HERC1, |
| | | TRPM7, NAMPT, TNRC6A, YRDC, CXCR3, FCGR3B, |
| | | PMAIP1, SOCS1, SLC25A36, GTF2I, LAMP2, NUAK2, |
| | | COMMD3-BMI1, LPIN1, MLLT11, B4GALT5, SATB1, |
| | | MYADM, FGL2, TC2N, CCL3L3, PROK2, RIC1, NPAT, |
| | | GPATCH2L, CCNG2, GOLGA8A, STX11, MAN2A1, |
| | | TFRC, UBE4A, UBE2D1, ZDHHC18, MYLIP, |
| | | MARCKS, CPEB2, ARRDC3, RBM47, PAFAH1B2, |
| | | MARCH1, SMC5, PTAFR, MORN2, ELL2, GOLGA8B, |
| | | HBA1, CD83, LCN2, CD69, LTF, TUBB1, CHORDC1, |
| | | 7-Mar, MCPH1, UGCG, TMEM68, HBM, OSBP2, |
| | | PDZK1IP1, NRGN, SLPI, CLIC3 |

Wherein, the different-level gene sets of the two various drugs were further compared. It was accidentally found that the second-level gene set (X2) of abiraterone and the second-level gene set (Y2) of enzalutamide had partially duplicate genes.

Finally, the results of the patients responded to various drugs were compared. As shown in FIG. 3C, individual scores of the patient responded to Abiraterone (Abi) and the patient responded to Enzalutamide (Enz) were compared and determined by the comparison module of the computer device. If the Score_{Abi}>Score_{Enz}, the recommendation module recommended the patient to choose Abiraterone as the preferred choice of the drug; otherwise. If the Score_{Abi}<Score_{Enz}, the recommendation module recommended the patient to choose Enzalutamide as the preferred choice of the drug.

For precise drug treatments for prostate cancer, the patient can be treated with the recommended second-generation hormone drug during the treatment of prostate cancer to seek good prognosis.

In order to enable a person skilled in the art to which the present invention belongs can understand the method of making and using the technique, the present invention has been described and exemplified in sufficient detail, however, various variations, modifications or improvements should be regarded as equivalent to the spirit and scope of the present invention.

A person skilled in the art to which the present invention belongs can easily understand and achieve the object of the present invention, and obtain the previously mentioned results and advantages. The sources, weight values or gene sets of physiological signals used in the present invention are exemplary in nature and are not intended to limit the scope of the present invention. The spirit of the present invention covers those skilled in the art and modifications or other uses arising from the production or use of the art, and is limited by the scope of the claims.

What is claimed is:

1. A system for indicating the use of second-generation hormone drugs in the treatment of prostate cancer, comprising:
an input device for receiving ex vivo biological samples obtained from a subject suffering from prostate cancer, and analyzing the ex vivo biological samples to generate at least one physiological signal (N) comprising genetic information of the subject suffering from prostate cancer; and
a computer device connected to the input device, wherein the computer device comprising:
a processor for receiving the at least one physiological signal (N), wherein the processor further comprises:
an analysis module for executing a calculation method to generate a first score (S1) and a second score (S2) by:
(i) comparing the at least one physiological signal (N) with a first-level gene set (X1) of patients suitable for a first second-generation hormone drug treatment to obtain a comparison value of (N, X1) and comparing the at least one physiological signal (N) with a second-level gene set (X2) of the patients suitable for the first second-generation hormone drug treatment to obtain a comparison value of (N, X2), then applying a first weight value (W1) to the comparison value of (N, X1) and a second weight value (W2) to the comparison value of (N, X2) to obtain a first score (S1) by using the calculation method, wherein the gene set X1 comprises the genes of NR4A1, ARHGEF10L, TNNT1, CPAMD8, CYP2S1, PLA2G7, CLEC10A, CD1C, ATF3, FCERIA, PLD4, the gene set X2 comprises the genes of SHROOM1, OSM, CD83, STAB1, UCN, AC106782.1, ANKRD28, CD69, FCHO2, CCR3, CPA3, CCR1, IFNG, OXER1, GAS6, KCNC3, ZNF385A, ELANE, BPI, FAM109A, LCN2, AZU1, DEFA3, MZB1, PCSK6, KLF4, IFI27, SIGLEC1, LGALS2, LRP1, PLXNB2, GATA2, FOSB, ZNF703, HISTIHIC, NUCB2, JUN, Clorf54, CDC42EP1, LILRB4, TUBB2A, CD24, RHOB, ITGB3, TGFBI, AHSP, ZNF358, and
(ii) comparing the at least one physiological signal (N) with a first-level gene set (Y1) of patients suitable for a second second-generation hormone drug treatment to obtain a comparison value of (N, Y1) and comparing the at least one physiological signal (N) with a second-level gene set (Y2) of the patients suitable for the second second-generation hormone drug treatment to obtain a comparison value of (N, Y2), then applying the first weight value (W1) to the comparison value of (N, Y1) and the second weight value (W2) to the comparison value of (N, Y2) to obtain a second score (S2) by using the calculation method, wherein the gene set Y1 comprises the genes of TMF1, SLC4A1, ASH1L, ARMC2, STXBP5, and the gene set Y2 comprises the genes of SOD2, AQP9, FAM174A, SRSF11, ZBTB11, CDC42EP3, CDK17, NFIL3, GCA, HBG2, HEATR5B, MANF, TTC3, XAF1, APOBEC3A, STRN3, HBQ1, WSB1, TNFAIP3, ZDHHC17, MYSM1, ZMAT1, DDX3Y, CPEB4, ACSL1, FAM156A, ERBIN, WDR44, HBB, SLC39A10, AL132780.3, FPR2, TREM1, RFX2, ATRX, TNIK, PCMTD2, TMEM127, PLCG1, ZBTB38, PHC3, ANKRD28, CXCR2, DYNCILI2, RNF103, EARS2, RNF19B, TLR8, HELZ2, ZNF518A, SLC38A2, MPZL1, CRLF1, DNAJC25-GNG10, RBL2, GOS2, CREBZF, LEMD3, TRAPPC10, MAP4K5, MGAM, PIK3R4, SCYL2, FAM198B, SDCBP, EIF5, PIGA, TAF1, KRR1, HIF1A, TLR4, GPD2, TCP11L2, PTP4A1, SREK1, ARID4B, KCTD12, TNFAIP8L2, TOB1, HBA2, BIRC3, PIKFYVE, UTRN, CDC14A, FAM76B, SENP7, GPR171, NKTR, GOLGA8N, NFKBIZ, GNS, PTGS2, ATXN7, ITGA6, PRF1, TNFRSF10C, RNPC3, KLF9, FAM91A1, PDK4, KIAA1109, ZNF548, HERC1, TRPM7, NAMPT, TNRC6A, YRDC, CXCR3, FCGR3B, PMAIP1, SOCS1, SLC25A36, GTF2I, LAMP2, NUAK2, COMMD3-BMI1, LPIN1, MLLT11, B4GALT5, SATB1, MYADM, FGL2, TC2N, CCL3L3, PROK2, RIC1, NPAT, GPATCH2L, CCNG2, GOLGA8A, STX11, MAN2A1, TFRC, UBE4A, UBE2D1, ZDHHC18, MYLIP, MARCKS, CPEB2, ARRDC3, RBM47, PAFAH1B2, MARCH1, SMC5, PTAFR, MORN2, ELL2, GOLGA8B, HBA1, CD83, LCN2, CD69, LTF, TUBB1, CHORDC1, 7-Mar, MCPH1, UGCG, TMEM68, HBM, OSBP2, PDZKIIP1, NRGN, SLPI, CLIC3;
a comparison module connected to the analysis module for comparing the first score (S1) and the second score (S2) to obtain a comparison result; and
a recommendation module connected to the comparison module for receiving the comparison result and outputting an indication, wherein an indication to take the first second-generation hormone drug is given when the first score (S1) is higher than the second score (S2), or an indication to take the second second-generation hormone drug is given when the first score (S1) is lower than the second score (S2).

2. The system of claim 1, wherein the recommendation module further connected to an electronic medical record system to output a prescription to the electronic medical record system according to the given indication.

3. The system of claim 1, wherein the recommendation module further connected to an automated dispensing cabinet connected to dispense the second-generation hormone drug according to the given indication.

4. The system of claim 1, wherein the first second-generation hormone drug is a compound of an androgen receptor blocker, and the second second-generation hormone drug is a compound of an androgen synthesis inhibitor.

5. The system of claim 1, wherein the calculation method comprises at least one algorithm.

6. The system of claim 4, wherein the at least one algorithm comprises Euclidian Distance, Manhattan Distance, Hamming Distance, Cosine similarity algorithm or Jaccard Similarity coefficient.

7. The system of claim 5, wherein the algorithm is the Jaccard Similarity (JS) coefficient.

8. The system of claim 6, wherein the first score (S1) and the second score (S2) are calculated by the following equation:
Score_{drug}=JS_{drug gene set 1}×W₁+JS_{drug gene set 2}×W₂.

9. The system of claim 1, wherein the first weight value (W1) and the second weight value (W2) range from 0 to 1.

10. The system of claim 7, wherein the first weight value (W1) is 1, and the second weight value (W2) is 0.8.

11. The system of claim 1, wherein the computer device further comprises a storage module for storing the at least one physiological signal (N), the comparison result and the indication.

12. A method for indicating the use of second-generation hormone drugs in the treatment of prostate cancer, comprising:
receiving ex vivo biological samples obtained from a subject suffering from prostate cancer by an input device, and analyzing the ex vivo biological samples to generate at least one physiological signal (N) comprising genetic information of the subject suffering from prostate cancer and sending the at least one physiological signal (N) to a processor;
comparing the at least one physiological signal (N) with a first-level gene set (X1) of patients suitable for a first second-generation hormone drug treatment to obtain a comparison value of (N, X1) and comparing the at least one physiological signal (N) with a second-level gene set (X2) of the patients suitable for the first second-generation hormone drug treatment to obtain a comparison value of (N, X2) by an analysis module of the processor, then applying a first weight value (W1) to the comparison value of (N, X1) and a second weight value (W2) to the comparison value of (N, X2) to obtain a first score (S1) by using a calculation method, wherein the gene set X1 comprises the genes of NR4A1, ARHGEF10L, TNNT1, CPAMD8, CYP2S1, PLA2G7, CLEC10A, CD1C, ATF3, FCER1A, PLD4, the gene set X2 comprises the genes of SHROOM1, OSM, CD83, STAB1, UCN, AC106782.1, ANKRD28, CD69, FCHO2, CCR3, CPA3, CCR1, IFNG, OXER1, GAS6, KCNC3, ZNF385A, ELANE, BPI, FAM109A, LCN2, AZU1, DEFA3, MZB1, PCSK6, KLF4, IFI27, SIGLEC1, LGALS2, LRP1, PLXNB2, GATA2, FOSB, ZNF703, HIST1H1C, NUCB2, JUN, Clorf54, CDC42EP1, LILRB4, TUBB2A, CD24, RHOB, ITGB3, TGFBI, AHSP, ZNF358, and
comparing the at least one physiological signal (N) with a first-level gene set (Y1) of patients suitable for a second second-generation hormone drug treatment to obtain a comparison value of (N, Y1) and comparing the at least one of the physiological signals (N) with a second-level gene set (Y2) of the patients suitable for the second second-generation hormone drug treatment to obtain a comparison value of (N, Y2) by the analysis module of the processor, then applying the first weight value (W1) to the comparison value of (N, Y1) and the second weight value (W2) to the comparison value of (N, Y2) to obtain a second score (S2) by using the calculation method, wherein the gene set Y1 comprises the genes of TMF1, SLC4A1, ASH1L, ARMC2, STXBP5, and the gene set Y2 comprises the genes of SOD2, AQP9, FAM174A, SRSF11, ZBTB11, CDC42EP3, CDK17, NFIL3, GCA, HBG2, HEATR5B, MANF, TTC3, XAF1, APOBEC3A, STRN3, HBQ1, WSB1, TNFAIP3, ZDHHC17, MYSM1, ZMAT1, DDX3Y, CPEB4, ACSL1, FAM156A, ERBIN, WDR44, HBB, SLC39A10, AL132780.3, FPR2, TREM1, RFX2, ATRX, TNIK, PCMTD2, TMEM127, PLCG1, ZBTB38, PHC3, ANKRD28, CXCR2, DYNCILI2, RNF103, EARS2, RNF19B, TLR8, HELZ2, ZNF518A, SLC38A2, MPZL1, CRLF1, DNAJC25-GNG10, RBL2, GOS2, CREBZF, LEMD3, TRAPPC10, MAP4K5, MGAM, PIK3R4, SCYL2, FAM198B, SDCBP, EIF5, PIGA, TAF1, KRR1, HIF1A, TLR4, GPD2, TCP11L2, PTP4A1, SREK1, ARID4B, KCTD12, TNFAIP8L2, TOB1, HBA2, BIRC3, PIKFYVE, UTRN, CDC14A, FAM76B, SENP7, GPR171, NKTR, GOLGA8N, NFKBIZ, GNS, PTGS2, ATXN7, ITGA6, PRF1, TNFRSF10C, RNPC3, KLF9, FAM91A1, PDK4, KIAA1109, ZNF548, HERC1, TRPM7, NAMPT, TNRC6A, YRDC, CXCR3, FCGR3B, PMAIP1, SOCS1, SLC25A36, GTF2I, LAMP2, NUAK2, COMMD3-BMI1, LPIN1, MLLT11, B4GALT5, SATB1, MYADM, FGL2, TC2N, CCL3L3, PROK2, RIC1, NPAT, GPATCH2L, CCNG2, GOLGA8A, STX11, MAN2A1, TFRC, UBE4A, UBE2D1, ZDHHC18, MYLIP, MARCKS, CPEB2, ARRDC3, RBM47, PAFAH1B2, MARCH1, SMC5, PTAFR, MORN2, ELL2, GOLGA8B, HBA1, CD83, LCN2, CD69, LTF, TUBB1, CHORDC1, 7-Mar, MCPH1, UGCG, TMEM68, HBM, OSBP2, PDZK1IP1, NRGN, SLPI, CLIC3;
comparing the first score (S1) and the second score (S2) to generate a comparison result by using a comparison module of the processor;
receiving the comparison result, and giving an indication by a recommendation module to recommend the subject suffering from prostate cancer to take the first second-generation hormone drug when the first score (S1) is higher than the second score (S2) or giving an indication to recommend the subject suffering from prostate cancer to take the second second-generation hormone drug when the first score (S1) is lower than the second score (S2), and administering the recommended second-generation hormone drug to the subject suffering from prostate cancer for prostate cancer treatment.

13. The method of claim 12 wherein the first second-generation hormone drug is a compound of an androgen receptor blocker, and the second second-generation hormone drug is a compound of an androgen synthesis inhibitor.

14. The method of claim 12, wherein the calculation method comprises at least one algorithm.

15. The method of claim 14, wherein the at least one algorithm comprises Euclidian Distance, Manhattan Distance, Hamming Distance, Cosine similarity algorithm or Jaccard Similarity coefficient.

16. The method of claim 15, wherein the algorithm is the Jaccard Similarity (JS) coefficient.

17. The method of claim 16, wherein the first score (S1) and the second score (S2) are calculated by the following equation:
Score_{drug}=JS_{drug gene set 1}×W₁+JS_{drug gene set 2}×W₂.

18. The method of claim 12, wherein the first weight value (W1) and the second weight value (W2) ranges from 0 to 1.

19. The method of claim 17, wherein the first weight value (W1) is 1, and the second weight value (W2) is 0.8.

20. The method of claim 12, wherein the computer device further comprises a storage module for storing the at least one physiological signal, the comparison results and the recommendation indication.
